(19) United States
(12) Patent Application Publication
(10) Pub. No.: US 2006/0233776 A1
(43) Pub. Date: Oct. 19, 2006

(54) C1-inh as a drug for treating viruses pathogenic to humans

(76) Inventors: Norbert Heimburger, Marburg (DE); Peter Gronski, Marburg (DE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/568,687

(22) PCT Filed: Aug. 13, 2004
(86) PCT No.: PCT/EP04/09085

§ 371 Date: Feb. 17, 2006

(30) Foreign Application Priority Data
Aug. 19, 2003 (DE) 10338533.9
Nov. 25, 2003 (DE) 103355408.4

(51) Int. Cl. A61K 38/43

(52) U.S. Cl. 424/94.1

(57) ABSTRACT

The C1 esterase inhibitor (C1-INH) from human plasma is a sialic-acid-containing glycoprotein which can bind other glycoproteins and glycolipids which may be components or even membrane components of microoganisms of viral and bacterial origin. In accordance with the invention, this binding can inhibit the penetration of certain viruses into target cells.

The C1 esterase inhibitor (C1-INH) from human plasma is a sialic-acid-containing glycoprotein which can bind other glycoproteins and glycolipids which may be components or even membrane components of microoganisms of viral and bacterial origin. In accordance with the invention, this binding can inhibit the penetration of certain viruses into target cells.

The interaction between HIV and C1-INH has already been described and is used by the authors to separate HIV from a liquid (EP 0 966 976 A1/Centeon Pharma GmbH) or, using a specially modified C1-INH molecule, to block the infectivity of HIV (EP 0969017 A1/Centeon Pharma GmbH).

The first evidence has now been found that C1-INH is capable of reacting with influenza virus (IV) particles. The sialic groups of the considerable C1-INH carbohydrate fraction could be involved in this interaction as a multivalent receptor for viral haemagglutinin membrane components of, for example, orthomyxoviruses (such as influenza A and B viruses) or paramyxoviruses (e.g. parainfluenza, mumps and measles viruses); C1-INH would thus become, in the mucosa, a constituent of an early acting defense system against infectious organisms. After all, the haemagglutinin molecule has, in the distal globular domain, a binding site which identifies and can bind sialic acid molecules on the surface of infectable cells. This component has not previously been described for the lentiviruses (retroviruses) HIV-1 and HIV-2; for this reason the C1-INH/HIV interaction should involve other groupings.

Surprisingly, however, evidence of a C1-INH/HIV interaction was also found, for example, after the treatment of C1-INH with neuraminidase.

Moreover, it is conceivable that C1-INH can inhibit the trypsin-like furin protease which splits the haemagglutinin precursor protein into two fragments linked by a disulfide bridge, a requirement for the fusogenic activity of the virus particles.

C1-INH is synthesized predominantly in the liver but also in epithellal cells and macrophages. Its plasma concentration is 25 mg/dl, the carbohydrate fraction 35%, of which 14% is slalic acid. Including a peptide fraction of 65%, its molecular weight is 104,000 (Haupt H. et al., 1970, Eur. J. Biochem 17: 254).

The high carbohydrate fraction above all determines the occurrence and interactions with lectins. The multifunctional protein is found in the mucosa of the respiratory tract as a first barrier to infectious agents.

Functionally, C1-INH belongs to the class of serine proteinase inhibitors. Its spectrum of activity includes regulation of contact factors in haemostasis: F XI a, F XII a, F XII fragment and the plasma kallkrein associated with it. In addition, C1-INH controls, via the inhibition of C1 esterase, the classic complement activation pathway (Heimburger N., 1975, Proteinase inhibitors of human plasma—their properties and control functions. In: Proteases and Biological Control; ed. Reich E., et al., 367; Cold Spring Harbor Symp.; Heimburger N., 1994, Haemostasologle 14 (1): 1). Since it is the only complement inhibitor produced by the body, it plays a major clinical role, particularly since it is known that several fulminant disease processes occur via activation systems controlled by C1-INH. This gives rise to consumption of the inhibitor which often can be offset only by replacing it. That applies to the angioneurotic syndrome (after Quincke, 1882; now called anglo-oedema) which is caused by a congenital deficiency of functional C1-INH, and to a number of other indications which have been added in recent years. The plasma-fractionating industry has responded to this need by manufacturing highly purified C1-INH concentrates. Recombinant human C1-INH is also available from several sources (Davis A. E. et al., 1992 Natur Genetics: 1: 354); at present, however, this product does not contain a carbohydrate fraction which corresponds to the molecule occurring in human plasma and which for this reason cannot possess certain functions of the carbohydrate fraction of plasma-based C1-INH.

C1-INH has such broad therapeutic importance mainly because it regulates and limits the inflammation process locally, however it is caused. This applies in particular to the mucosa. Contact factors can react sensitively to any change in endothelial surfaces, e.g. by activating haemostasis, fibrinolysis and the complement system. This gives rise, among other things, to bradykinin-type vasoactive peptides which influence vascular tone. In the capillary region this can lead to dilation and increased permeability which can cause oedema or even capillary leak syndrome (Eisele B. et el., 1994, “Die gelben Hefte”, vol. XXXIV, Issue 4: 162). If C1-INH does not limit this process at an early stage by neutralization of the proteinases involved, it can spread to the organs in the form of general inflammation. That explains why C1-INH is used in so many syndromes such as capillary leak and circulatory shock (EPA 0586 909 A 2/Behringwerke A G), sepsis and septic shock [EP 0620406 B 1/Behringwerke A G) and in the extracorporeal circulation (DE-A-4227762/Behringwerke A G).

What is new is that C1-INH not only inactivates proteinases involved in infection and inflammation but also interacts with viruses distinguishable from HIV by means of molecular regions other than the ones involved there and potentially neutralizes them. This reactivity could for example have a special importance in acute mumps or measles infections. C1-INH is therefore a suitable means of alleviating the severity and consequences of acute measles or mumps infections. Neutralization evidently does not occur with the unchanged, native C1-INH molecule in relation to human immunodeficiency viruses (EP 0 969 017 A 1/Centeon Pharma GmbH), which is why a specific modification was proposed.

Finally, there is evidence that the mucus of healthy individuals contains epithellal-cell-based glycoproteins which, “like cell receptors, bind virus particles and prevent infection”. (Lange W., Vogel G., F. Uphoff H., 1999, Influenza Virologie, Epidemiologle, Klinlk, Therapie und Prophylaxe; Blackwell Wissenschafts-Veriag). Virus particles means influenza viruses (IV). Since many proteins were first isolated from human plasma at the Behringwerke; we tried to identify the glycoprotein(s).

We used IV for the model studies. Since we did not want to work with live viruses, we selected a widely used vaccine which is declared as a chemically inactivated subunit, mixed vaccine containing 45 μg haemagglutinin in an application volume of 0.5 ml. (Begrivac®). We could not detect any immunogen in the IV suspension using either Ouchterlony's agar gel diffusion technique or precipitating anti-IV antibodies. We were however able to do so after making a small modification (FIG. 1); the central hole in the agar plate was initially filled with the vaccine and 24 hours later with a C1-INH solution in order to dissolve the IV antigens out of their interactions in the vaccine and to determine them.

To this end the punched holes arranged in a circle around the central hole were filled separately with pooled human plasma and 2 polyclonal human immunoglobulin concentrates (Beriglobin® P) containing antibodies to IV. All showed, probably dependent on the antibody titre, 2 more or less sharply defined precipitates. The vaccine thus contains at least 2 antigen populations which both bind C1-INH (as is visible in FIG. 1) and differ in their molecular-weight-dependent rate of diffusion. Detection of antigens from the vaccine is concentration-dependent and begins at 62.5 μg/ml C1-INH with 90 μg/ml haemagglutinin. It is not possible to give more accurate information using this technique. The immunoprecipitates in the agar gel are usually so fine that they are visible only after staining with Coomassie brilliant blue.

In immunoelectrophoresis, the subunit vaccine migrates heterogeneously in the β-to α2-globulin region without forming a typical precipitate arc (FIG. 2a). After the addition of C1-INH in 20-fold excess, the IV antigens concentrate in the α2 region, trailing off at the site of application. The material precipitates over the entire region even with antibodies to C1-INH, in the form of an extension to the sickle-shaped precipitate formed by the C1-INH alone (FIG. 2b).

From the findings it follows that the subunit vaccine, because of its splitting and chemical inactivation, consists of components with differing electrophoretic mobility which all bind to C1-INH and all have the same suitable acceptor. The main component, which probably contains haemagglutinins, forms complexes with α2-globulin mobility. In addition, several higher-molecular-weight, more slowly migrating complexes and aggregates are visible. The striking thing is that the C1-INH/IV antigen complexes with antibodies to C1-INH are much more clearly visible than with anti-IV antibodies. This phenomenon may arise because C1-INH, which is potentially multivalent as regards IV, carries a large number of terminal sialic acid groups via which it can bind and perhaps even cross-link IV and other haemagglutinin-containing viruses, as a result of which epitopes recognizable by anti-IV antibodies could be blocked.

It was important to investigate the significance of sialic acid groups for the C1-INH/IV antigen interaction. To this end, treatment with test neuraminidase (test NA, Dade Behring) from Vibrio colerae (VC) was performed at pH 7.0 (optimum pH at about 5.5-6.0); this was done either before or after adding the vaccine to the C1-INH. Immunoelectrophoresis illustrates that the neuraminidase treatment (FIG. 3b) or the C1-INH/IV antigen complex formation reduce the mobility of the C1-INH to a comparably large extent (FIG. 3d) and that the two effects are additive (FIG. 3c), evidently without having any effect on the sequence of enzyme treatment (see FIG. 3c with 3f). It is also worth noting that C1-INH binds IV antigens even after neuraminidase treatment (FIG. 3c) and gives off the preformed complex neuraminic acid. This finding is consistent with the reversibility of C1-INH/IV antigen complexing and with the existence of binding sites other than haemagglutinin on the C1-INH.

One possible candidate was NA, and specifically its active centre (AC). To find out if this was the case, we performed binding studies in the presence of a specific, synthetic NA inhibitor which sterically blocks the AC of IV-NA, thus preventing IV replication: Tamiflu® (Roche).

FIG. 4 shows the immunoelectrophoretic characterization of C1-INH (4a) and of the complex with IV vaccine (4b) which, surprisingly, does not form in the presence of Tamiflu® (4c). If an NA isolated from VC (VC-NA) is added to this sample set-up (4c), the antiserum to C1-INH which was used for this entire series of experiments shows a precipitate which runs into the site of application; it is identical to the VC-NA complex (4d). This is also seen in the set-up (4e) in which NA from IV and VC were preincubated together before C1-INH and Tamiflu® were added. As the result shows, the soluble bacterial NA binds better to C1-INH than the particulate IV-NA.

Conclusion: If the AC of IV-NA is blocked by Tamiflu®, the complex with C1-INH does not form. Both inhibitors evidently have the same target on the IV, but the synthetic inhibitor binds the AC of NA with higher affinity. And that should also be the 2nd binding site for C1-INH. On the basis of the reaction type, this bond should be stronger than that of the haemagglutinins to the neuraminic acid groups of the C1-INH.

According to these results, C1-INH acts as a competitive NA inhibitor, it is not specific like Tamiflu® but inhibits the NA from viruses and bacteria without regard to species (FIGS. 4d and e). What we know about the importance of NA inhibitors we know in part from the influenza viruses: they bind viruses, transport them—probably in a noninfectious form—present them to the immune cells and prevent reproduction. This type of inhibitor was, far-sightedly, classified by Bumet as a “competitive poison” as long ago as 1948 (Gubareva L V et al., Lancet 2000; 355: 827).

As regards the properties and identity of the inhibitor, with C1-INH there is no doubt if human plasma with and without the addition of VC-NA is tested by immunoelectrophoresis with an antiserum to C1-INH, the VC-NA/C1-INH complex is found directly at the site of application; an antiserum to the inter-α-trypsin inhibitor shows that the homologous protein also contains neuraminic acid: after the addition of NA, it migrates little, but does so much more slowly (FIG. 5).

Tests with citrated or EDTA plasma underlife the importance of Ca²⁺ ions. Immuno-electrophoresis in the presence of these complexing agents does not yield any direct evidence of visible precipitates with reduced mobility (FIG. 6). This is shown above all by comparison with the plasma-free system (FIG. 7). It is however noticeable that the addition of the vaccine leads to weaker formation of precipitates characteristic of the free (non-complexed) C1-INH; this points to a consumption reaction which is not initially visible.

However, after staining the agar gels it becomes clear that C1-INH/lV antigen complexes can even be formed in the citrated medium in which predominantly free, non-protein-bound Ca²⁺ ions are complexed; this does not happen at all in the presence of EDTA (FIGS. 7, 3a-c).

It is noticeable that the citrated plasma already contains IV-C1-INH complexes in situ. The precipitate becomes stronger after the addition of IV, and after incubation at 37° C. It is also apparent that it communicates with the free C1-INH (FIGS. 7, 2c).

We had the first evidence of the existence and incidence of these complexes in citrated plasma even earlier (FIG. 8): they are formed as variable amounts of precipitate, comparable to a plaque, when the agar gel is developed with antisera to C1-INH and/or to IV antigens. If IV vaccine is added to a citrated plasma of this kind, the concentration of free C1-INH decreases, whereas the plaque gets bigger.

A substantial addition to our findings was that the formation of IV/C1-INH complexes is reversible and dependent on Ca²⁺ ions: thus, complexes are formed in an IV-containing EDTA plasma if it is dialysed against a citrate solution with 5 mM CaCl₂ and 0.9% (m/v) NaCl.

Surprisingly, IV antigens can be detected in human plasma by immuno-electrophoresis. In 4 different human plasma pools investigated by us, we found, with human anti-IV antibodies, a punctiform slowly diffusing antigen in the α2-globulin region (FIG. 8) and sometimes, further towards the anode, a soft, sickle-shaped precipitate (not visible in FIG. 8). The origin of the two IV antigens, which clearly circulate as a complex with C1-INH in the blood, is unclear, either they are from an influenza infection or they are the result of vaccination. At any rate, according to their electrophoretic mobility, they could be processing product of IV.

After the addition of subunit influenza vaccine at the same plasma pool, a large, intensely coloured precipitate appears anodally to the site of application (FIG. 8a, bottom); it can be attributed to antigens present in the vaccine which partially correspond in their charge-dependent diffusion characteristics to the IV antigens already present in the plasma. That can be seen even more clearly in immuno-electrophoresis of the two plasma pool samples which were developed with an antiserum to C1-INH (FIG. 8b). Wherever IV antigen is present in FIG. 8a. C1-INH is also found, together with, of course, the sickle-shaped precipitates of C1-INH which has not bound IV antigen. It is noticeable that the C1-INH is reduced in the vaccine-enriched plasma; it is obviously bound by the vaccine (FIG. 8b bottom).

This view is also supported by the high colour intensity characteristic of IV/C1-INH complexes which is what makes possible their detection in high dilution in human plasma.

It is quite conceivable that the C1-INH/IV complexes can also be detected using other technologies: for examples by a sandwich ELISA method by means of specific capture antibodies immobilized on a suitable matrix against one of the two components and using a second, marked antibody against the other component. The classic, relatively simple gel precipitation methods used have however moved sufficient.

Diagnostically, the interaction of C1-INH with pathogenic agents could be potentially important, because the quantitative detection of, for example, C1-INH bound to virus particles or virus constituents might correspond to a particular infection status.

Our investigations were prompted by the observation mentioned earlier (by Lange, W. et al.; see above) that the mucous membrane contains glycoproteins which form an initial barrier in the defense against infections; they bind IV particles and thus inhibit infection.

We have identified C1-INH as one of the glycoproteins and, after tests with this model vaccine, can confirm that it has the ability to bind IV:

1. IV antigens as inhibitor complexes with varying diffusion capabilities can be detected concentration-dependently from an IV vaccine with C1-INH by means of suitable antibodies (FIG. 1).

2. In the electrical field used in immunoelectrophoresis, the vaccine migrates from the site of application to the α2-globulin region. In doing so, it dissociates into many components of varying mobility (FIG. 2). After the addition of C1-INH the material is enriched in a typical precipitate of the kind that can be produced with anti-IV antibodies. The interactions of the mixed and subunit vaccines with C1-INH become visible with an antiserum to C1-INH. With the resultant increased detection sensitivity it can be seen that C1-INH/IV antigen complexes of varying mobility must lie along the entire migration path.

3. No conspicuous heterogeneities are found in the plasma, even after the addition of vaccine. The complexing of IV components by C1-INH may be an explanation for this (FIG. 8).

4. In each of 4 pools of citrated human plasma we found one complex of C1-INH and one of IV antigens. In the electric field it migrates faster than the vaccine added for comparison purposes. We have evidence that there are more complexes of this kind which are smaller and more mobile than the vaccine (see above) but are at the limit of detection.

5. As we have shown, IV have 2 binding sites for C1-INH: the haemagglutinins and the AC of NA. Both bind to the neuraminic acid, groups of C1-INH; the stronger bond is likely to be from the AC of NA; it is formed first; in a solution containing IV and C1-INH, the splitting of neuraminic acid residues is seen to occur much delayed—it is recognizable from the decrease in electrophoretic mobility—and often only from 37° C. onwards. This too shows that IV-NA is inhibited by C1-INH. Applied to physiological conditions it can be concluded from this that IV on the mucous membrane is not only bound but also inactivated by C1-INH. It can thus also be assumed that the IV antigen found in the pool plasma by immunoelectrophoresis is not infectious, particularly as we have observed virus complexes only in plasmas which also contained antibodies to the homologous virus. This defense mechanism is impressive because it guarantees that no infectious material gets into circulation. Two plasma proteins see to that the C1-INH and antibodies to the virus; the two molecules “cooperate”: the C1-INH/IV complex is for example dissociated from the homologous virus antibody and the virus is taken over. These complexes, which probably exist in equilibrium, and their prolonged presence could explain the life-long immunity known to be conferred by, for example, vaccination against measles (Pschyrembel, Klinisches Wörterbuch 257th edition 1994; de Gruyter, p. 941) and against smallpox (Hammarlund E et al.; Nature Medicine 2003; 9: 1131).

Since NA are widespread, particularly in infectious agents, and specifically in viruses and bacteria, the principle of binding, inactivation and immune defense described for IV could have broad biological applications and therapeutic ones.

These properties make C1-INH an important element in the defense against micro-organisms such as infectious viruses. Since the principle is simple, effective and very promising and C1-INH is available as a highly purified protein for intravenous administration, it is an alternative to or at least an addition to the usual adjuvant-vaccine combinations; it can above all be used in cases where it is known that the infectious agent contains neuraminidase (e.g. bacteria) or haemagglutinin and neuraminidase (virus particles such as IV, paramyxoviruses, rotaviruses and perhaps even SARS) as a constituent of its membrane. Given the properties of C1-INH, extravascular administration via the mucous membranes is both useful and conceivable.

### FIGURES

FIG. 1 Detection of IV-specific antigens in an IV vaccine in the Ouchterlony agar gel diffusion test
Middle; IV vaccine (90 μg/ml) and after 24 h
C1-INH solution (125 μg/ml)
In the semi-circle from left;
Pooled citrated human plasma
Anti-C1-INH serum
Immunoglobulin concentrate (batch 1)
Immunoglobulin concentrate (batch 2);

FIG. 2 Characterization of the IV vaccine and IV/INH complex by immunoelectrophoresis in agarose
a) Trough: lmmunoglobulin concentrate (batch 1)
b) Trough: Anti-C1-INH serum
   1. IV vaccine (5 μg),
   2. IV vaccine (5 μg) plus C1-INH (100 μg)
   3. C1-INH (100 μg)
All samples in a final volume of 100 μl
Application volume 10 μl, diffusion time 17 h

FIG. 3 Action of neuraminidase on C1-INH and the binding of IV antigens
In each case 500 μl C1-INH (250 μg/ml) were treated with 10 μl test neuraminidase (Dade Behring, QRKD 253670)
   and/or
50 μl IV vaccine (4.5 μg):
a) C1-INH,
b) C1-INH plus neuraminidase,
c) C1-INH plus neuraminidase and IV vaccine,
d) and e) C1-INH plus IV vaccine,
f) C1-INH/IV complex plus neuraminidase
Volume made up with physiol. NaCl; incubation for 15 min at room temperature
Troughs: Anti-C1-INH serum, application volume 10 μl: diffusion time 17 h

FIG. 4 Inhibition of IV vaccine binding to C1-INH by a synthetic, specific NA inhibitor: Tamiflu® (Roche)
a) C1-INH (250 μg/ml, 100 μl)
b) IV vaccine (50 μl) plus C1-INH
c) IV vaccine preincubated with Tamiflu® (10 μg/100 μl) plus C1-1NH
d) IV vaccine preincubated Tamiflu® plus VC-NA and C1-INH
e) IV vaccine preincubated with VC-NA plus Tamiflu® and C1-INH
Troughs: Anti-C1-INH serum
All mixtures made up to the same volume; preincubated overnight at room temperature and, before immunoelectrophoresis, for 2 h at 37° C. Application volume 10 μl, diffusion time 17 hr

FIG. 5 Characterization of the VC-NA/C1-INH complex formed after the addition of VC-NA (test neuraminidase) to citrated human plasma
a) Human plasma diluted 1:2 with phys. NaCl
b) Human plasma diluted 1:2 with test neuraminidase
Troughs: top ant-C1-INH serum
   Bottom anti-inter-α-trypsin inhibitor serum
Application volume 10 μl, diffusion time 17 h

FIG. 6 Binding of IV antigen to isolated C1-INH (1), to C1-INH in citrated human plasma (2) and in EDTA plasma (3)
1 0.5 ml C1-INH a) plus 50 μl physiol. NaCl
   b) plus 50 μl IV vaccine
   c)=b) 1 h incubated at 37° C.
2 0.5 ml citrated plasma a) plus 50 μl physiol. NaCl
   b) plus 50 μl IV vaccine
   c=b) incubated for 1 h at 37° C.
3 0.5 ml EDTA plasma a) plus 50 μl physiol. NaCl
   b) plus 50 μl IV vaccine
   c=b) incubated for 1 h at 37° C.
Troughs: Anti-C1-INH serum
Application volume: 10 μl, diffusion time 17 h

FIG. 7 The agar gels in FIG. 4 after staining with Coomassie brilliant blue

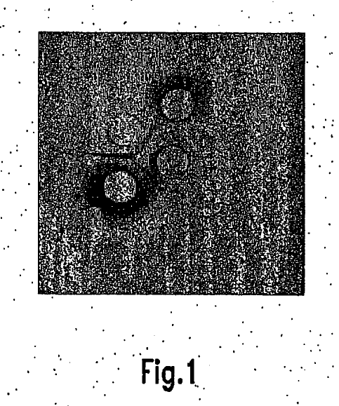

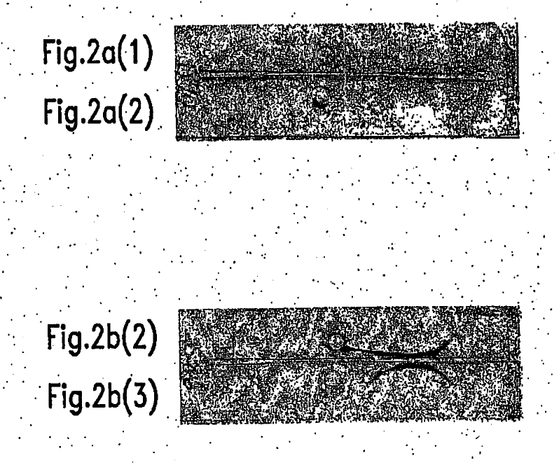

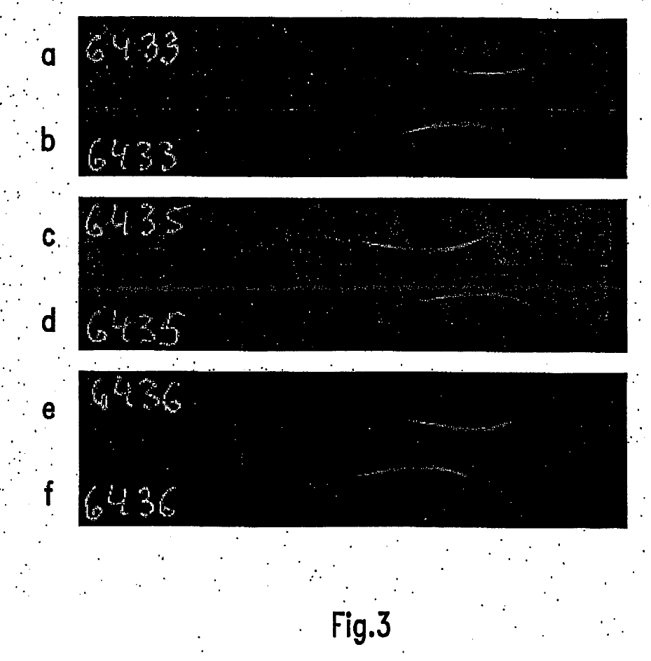

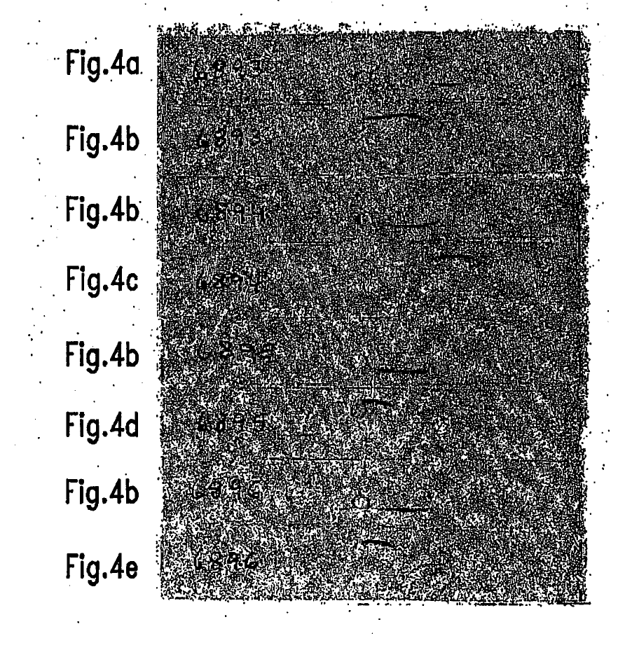

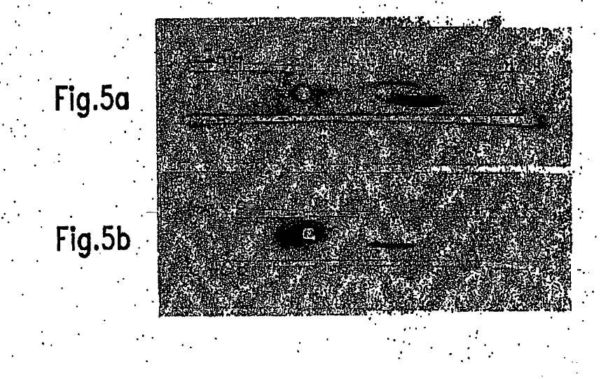

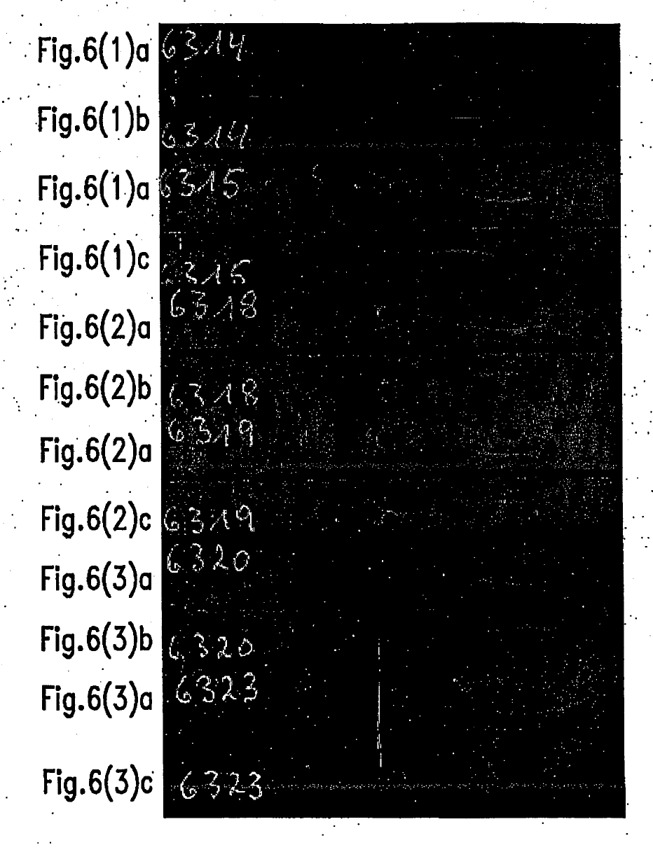

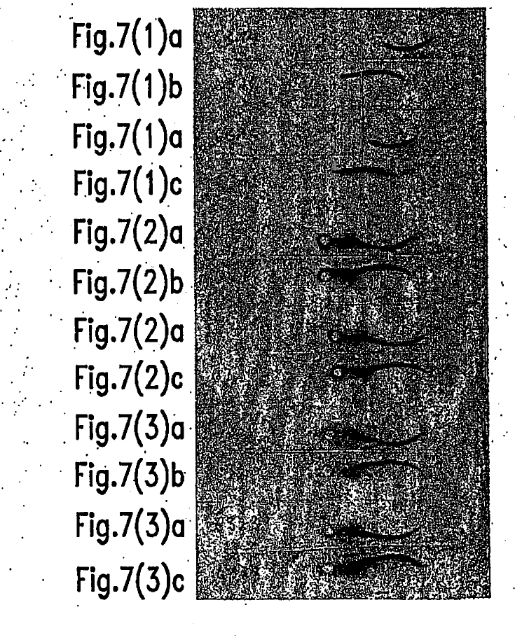

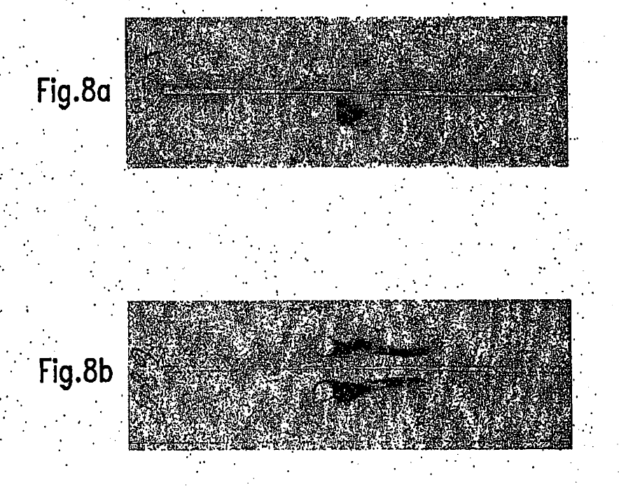

FIG. 8 Identification of the IV and its complex in citrated plasma by immunoelectrophoresis
a) Trough: Immunoglobulin concentrate
b) Trough: Anti-C1-INH serum
1. Pooled citrated human plasma
2. 2.5 μg IV vaccine in 50 μl pooled, citrated human plasma

1-6. (canceled)

7. A method of treating an infection caused by at least one of a virus and a bacterium comprising administering to a subject an effective amount of a composition comprising C1 esterase inhibitor, wherein the at least one virus or bacterium comprises at least one of haemagglutinin and neuraminidase.

8. The method of claim 7, wherein the infection is an acute infection.

9. The method of claim 7, wherein the virus is chosen from orthomyxovirus, paramyxovirus, and rotavirus.

10. The method of claim 9, wherein the orthomyxovirus is chosen from influenza A virus and influenza B virus.

11. The method of claim 9, wherein the paramyxovirus is chosen from parainfluenza, mumps virus, and measles virus.

12. The method of claim 7, wherein the bacterium is Vibrio cholerae.

13. The method of claim 7, wherein the composition is administered intravenously or mucosally.

14. The method of claim 7, wherein the subject is a human subject.

15. The method of claim 7, further comprising administering a vaccine against the virus or bacterium.

16. A method of modulating an immune response to a virus, to a bacterium, or to a vaccine against the virus or bacterium comprising administering to a subject a composition comprising C1 esterase inhibitor, wherein the virus or bacterium comprises at least one of haemagglutinin and neuraminidase.

17. The method of claim 16, wherein the virus is chosen from orthomyxovirus, paramyxovirus, and rotavirus.

18. The method of claim 17, wherein the orthomyxovirus is chosen from influenza A virus and influenza B virus.

19. The method of claim 17, wherein the paramyxovirus is chosen from parainfluenza, mumps virus, and measles virus.

20. The method of claim 16, wherein the bacterium is Vibrio cholerae.

21. The method of claim 16, wherein the composition is administered intravenously or mucosally.

22. The method of claim 16, wherein the subject is a human subject.

23. A method of detecting the presence of a virus or a bacterium in a sample comprising:
(a) obtaining a sample;
(b) exposing the sample to C1-INH, wherein the C1-INH binds to the virus or bacterium; and
(c) detecting the virus or bacterium bound to C1-INH
wherein the virus or bacterium comprises at least one of haemagglutinin and neuraminidase.

24. The method of claim 23, wherein the virus is chosen from orthomyxovirus, paramyxovirus, and rotavirus.

25. The method of claim 24, wherein the orthomyxovirus is chosen from influenza A virus and influenza B virus.

26. The method of claim 24, wherein the paramyxovirus is chosen from parainfluenza, mumps virus, and measles virus.

27. The method of claim 23, wherein the bacterium is Vibrio cholerae.

28. The method of claim 23, wherein the sample is obtained from a human.

29. The method of claim 28, wherein the sample is human plasma.

30. A method of isolating a bacterium or virus comprising binding C1-INH to the virus or bacterium, wherein the virus or bacterium comprises at least one of haemagglutinin and neuraminidase.

31. The method of claim 30, wherein the virus is chosen from orthomyxovirus, paramyxovirus, and rotavirus.

32. The method of claim 31, wherein the orthomyxovirus is chosen from influenza A virus and influenza B virus.

33. The method of claim 31, wherein the paramyxovirus is chosen from parainfluenza, mumps virus, and measles virus.

34. The method of claim 30, wherein the bacterium is Vibrio cholerae.

35. A composition for the treatment of an infection caused by a virus or bacterium comprising C1-INH and antibodies against the virus or bacterium, wherein the virus or bacterium comprises at least one of haemagglutinin and neuraminidase.
